(19) United States
(12) Patent Application Publication
(10) Pub. No.: US 2009/0060778 A1
(43) Pub. Date: Mar. 5, 2009

(54) METHOD OF MOLD REMEDIATION

(76) Inventors: Edward R. Close, Jackson, MO (US); Jacquelyn A. Close, Jackson, MO (US)

Correspondence Address: FULWIDER PATTON LLP, 6060 CENTER DRIVE, 10TH FLOOR, LOS ANGELES, CA, 90045, US

(21) Appl. No.: 11/849,039

(22) Filed: Aug. 31, 2007

(51) Int. Cl. A61L 9/015

(52) U.S. Cl. 422/4

(57) ABSTRACT

A method for remediation of mold in a building comprising the steps of investigating the building to determine a presence of unwanted mold, sampling said unwanted mold both inside and outside of said building to determine a species of said mold and an indoor to outdoor ratio of mold spores for said species, a spore concentration for said species, and a number of colony forming units for said species, diffusing essential oils into the building for a predetermined period of time, where said predetermined period of time is based on a determination of three measures of said species, ratio of indoor to outdoor mold spores, spore concentration, and number of colony forming units; and directly applying a household cleaner with essential oils to any mold-affected areas. Removal and preventative steps prevent the recurrence of the mold infestation.

### BACKGROUND OF THE INVENTION

All known types of mold are members of a very large family called fungi. Fungi include mold, mildew, mushrooms, slimes, yeasts, smuts, and crop rusts. The fungi kingdom includes some very important organisms, in terms of both their ecological and economic roles. Fungi break down dead organic material, and there are plants that could not grow without the symbiotic fungi that inhabit their roots and supply essential nutrients. Other fungi provide drugs such as antibiotics, as well as foods to eat, including mushrooms, truffles and morels, and fungi are also responsible for the bubbles in bread, champagne, and beer. Very adaptable, fungi can change in form and function depending on many factors. For instance, some mold species are generally non-toxic; however, when their space is invaded by other competing fungi species, these non-toxic molds can become toxic. They will produce toxins in order to kill or dissuade invaders. Unfortunately, molds also create many problems for humans.

There is virtually no place on the planet that is free of mold. Mold thrives everywhere from thirty thousand feet or more in the air to the deepest mine in the earth. It can survive without air, in freezing or boiling hot environments. It grows in the tropics, in the Arctic and Antarctic, and on the highest mountain. It truly thrives and flourishes in the same temperature range that defines the comfort zone for human beings.

The presence of mold, however, can present health issues to humans. In particular, mold found in dwellings and office buildings are responsible for various health problems that range from itching eyes, sneezing, and coughing to serious allergic reactions, asthma attacks, bleeding lungs, and even death. Researchers at Lawrence Berkeley National Laboratory, Indoor Environment Department, reported in the peer reviewed International Journal of Indoor Environment and Health (June, 2007) that approximately 4.6 million cases of asthma may be attributed to dampness and mold exposure in the home. They further stated that risks of exposure to dampness and mold in schools, offices, and institutional buildings are similar to those in the home, costing taxpayers $3.5 billion annually.

Further to the issue of mold's effects on living quarters, the Harvard University School of Public Health studied ten thousand homes in the United States and Canada and found that half of them had conditions of water damage and mold associated with a 50 to 100% increase in respiratory symptoms. A 1999 study by the Mayo Clinic found that 96% of the 37 million Americans who suffer from chronic sinusitis symptoms do so because of mold exposure. As a result, Americans spend billions of dollars every year on over-the-counter medications, doctors, and prescription medications to treat the symptoms of the cold-like illnesses, incurring billions of dollars annually in lost productivity, and experiencing significantly impaired quality of life.

In a study published in the American Journal of Managed Care, Vol. 6, No. 3, March, 2000, a group of scientists reported that at-work productivity losses associated with a diagnosis of allergic rhinitis “were estimated to range from $2.4 billion to $4.6 billion [per year].” Dr. Javed Sheikh, Harvard University Medical School, Division of Allergy and Inflammation, in an article entitled “Rhinitis, Allergic” updated Mar. 2, 2007, says, “Rhinitis is defined as inflammation of the nasal membranes and is characterized by a symptom complex that consists of any combination of the following: sneezing, nasal congestion, nasal itching, and rhinorrhea [runny nose]. The eyes, ears, sinuses, and throat can also be involved . . . The total direct and indirect cost of allergic rhinitis was recently estimated to be $5.3 billion per year.” Dr. Sheikh says the causes for allergic rhinitis include exposure to pollens and molds.

Environmental Health Perspectives (EHP), a peer-reviewed journal published by the National Institute of Environmental Health Sciences (NIEHS), Mar. 2, 2005, says, “Exposure to mold and dampness in homes as much as doubles the risk of asthma development in children.” And in a Mar. 31, 2005 release, EHP says, “Office workers in a northeastern U.S. building that had been damaged by water leaks over a period of years were more than twice as likely to suffer from wheezing or asthma, and over three times more likely to suffer from adult-onset asthma, compared with the general adult population.”

From 1992 to 1999 a cluster of at least sixteen infant deaths in the Cleveland, Ohio, area resulted from acute idiopathic pulmonary hemorrhaging (bleeding lungs). In each and every case, one of the commonalities found was exposure to the mold Stachybotrys chartarum, a deadly toxic mold. A clinical study currently being conducted by Case Western Reserve (study start: January, 1999; expected completion: February, 2010) for the NIEHS is collecting samples of secretion, blood, and urine from infants diagnosed with idiopathic pulmonary hemorrhage and analyzing these fluids for fungal spores and mycotoxins. In study details provided by the NIEHS, it states that another 138 cases of acute idiopathic pulmonary hemorrhaging in infants were identified nationwide during the four year period 1995 to 1999 and cites a CDC case-control study that found an association with water-damaged homes and the toxigenic fungus Stachybotrys chartarum.

Two of the predominant modes by which mold affects human health are spores and mycotoxins. Both can be airborne, and both can cause irritation and allergic response upon contact, as well as many other serious symptoms and diseases. Spores can be irritants and cause allergic reactions both on the surface of the skin and inside the human body. They can grow inside nasal passages, sinus cavities, bronchial passages and lungs, living on a combination of particulate matter, mucus, and tissue. Even dead mold spores can produce irritation, allergic reactions, and other health problems. Some mold species appear to produce only one toxin, while others are known to produce over one hundred. Some molds also produce compounds called synergizers, which enhance the effects of their toxins. When one smells mold, one is breathing microbial volatile organic compounds (MVOCs) produced by mold, which may be toxic. More than five hundred MVOCs produced by mold species have been identified so far.

The study of toxins produced by mold species is still in its infancy. Currently, hundreds of mycotoxins have been identified, and many more are suspected. It is likely that the number will eventually be in the thousands. Molds produce mycotoxins from other chemicals like polypeptides and amino acids, which they use in metabolism. MVOCs, on the other hand, are derived from alcohols, ketones, and hydrocarbons. Mycotoxins are not volatile and are attached to mold spores. MVOCs are gaseous at room temperature, mix easily with air, and impact our olfactory nerves.

Mycotoxins produced by the Aspergillus species are called aflatoxins. Stachybotrys chartarum produces three mycotoxins: Roridin E, Verrucarin J, and Satratoxin H³, and Stachybotrys spores have these toxins on their surfaces, probably to protect the spores in an environment made hostile by other, competing mold species. So, in the case of Stachybotrys, it is not only the odiferous MVOCs but inhaled spores that carry toxins into the breathing passages and into the lungs. Stachybotrys spores are sticky spores that are not normally found in air samples.

Direct comparison of prior art mold-remediation technologies is difficult due to the differing modes of application and methods for measuring results. For example, fogging with chemical fungicides provides a one-time shock treatment of entire rooms or confined spaces, while ultraviolet (UV) light continuously treats the air stream in the air-handling system of an office, school, hospital, or home. While fogging a chemical fungicide will impact mold-spore source colonies, there is little or no residual effect to prevent reinfestation. UV irradiation, on the other hand, provides continuous eradication of mold spores in the air stream but does nothing to eliminate the source colonies. UV installation is costly and does not eliminate mold that may enter a building through windows, doors, or any other avenue of ingress that is downstream of the UV generator and filter.

Bleach, long recommended for cleaning moldy surfaces, compares poorly with most other mold-eradication methods for the following reasons: (1). Sodium hypochlorite (NaClO), the active ingredient in bleach, is produced by infusing sodium hydroxide with chlorine gas, a deadly gas that is extremely harmful if breathed into the lungs. (2). If bleach is accidentally mixed with an acid-containing cleaner (such as many toilet-bowl cleaners, drain cleaners, lemon juice or vinegar), then deadly chlorine gas can be released. (3). Because it is so caustic and dangerous, household bleach contains only 3% to 6% sodium hypochlorite. The remaining 94% to 97% volume of household bleach is water. Therefore, using bleach or a bleach solution actually provides one of the three ingredients necessary for mold growth (water) and can allow for a quick rebound of mold growth within 24 hours or less. (4). Bleach kills mold on nonporous surfaces, but not on porous surfaces and does nothing to the spores that are in the air. When the sodium hypochlorite dissipates, usually within a few hours of application, then mold growth can rebound. Application of liquid bleach to porous, mold-infested materials (such as wood, wallpaper, sheetrock, and the grout between bathroom tiles) will bleach the spore colonies and kill mold spores, but not all spores will be killed, and even dead spores can cause allergy symptoms.

The cost for cleaning up and remediating mold, using the old, standard methods employed by most mold-remediation companies, is high. In 2001, insurance companies paid $1.3 billion dollars in claims for damages to property by mold. In 2002, they paid more than $3 billion in mold-related property damage claims. Recently, some states in the U.S. have begun introducing legislation that requires that insurance companies doing property and casualty business in their state provide limited coverage for mold-related claims. And many insurance companies continue to pay to repair damage caused by water leaks, freezing, and flooding (if covered in the property owner's policy), but they will not pay to remediate mold. Additionally, in some cases when a claim for mold cleanup or mold remediation is filed, the properties' address is entered into a database and that information follows the property in the future, even after the mold problem is remedied. This will negatively impact the value of that property and its salability forever.

While research has linked many serious health problems to toxic mold, and more links are being discovered every day, a cleanup method that is safe, not hazardous, and provides for eradication of mold spores on both porous and non-porous surfaces, as well as in the air, has not been used in conventional mold remediation practice. If one does not know whether one is dealing with a toxic mold or a benign mold, then one must treat every mold infestation as if it is dangerous to prevent the spread of toxic spores. If the treatment assumes the mold is benign, it may create health problems for the occupants and the persons performing the remediation, possibly spreading the problem throughout the building. And if the materials happen to be infested with a toxic mold species, then this type of action creates a far more serious and costly problem to clean up.

### SUMMARY OF THE INVENTION

The present invention is a method for dealing with the dangers and high cost of mold remediation in homes, offices, and other enclosed spaces with simple, safe, cost effective materials that are extremely effective in eliminating both source mold colonies as well as airborne mold spores. The invention is directed to steps for remediation centered around the dispersion of selected essential oils for continuous periods of twenty-four to seventy-two hours based on parameters derived from sampling and analysis of the extent of the mold infestation. Factors involved in the determination include the ratio of spores found indoors to spores found outdoors, the number of mold colony forming units, and whether the mold is toxic. These factors dictate the length of time and amount of essential oils diffused into the enclosures to eradicate the mold.

When the term essential oils is used herein, it refers to the lipid (oil) soluble portion of the volatile, aromatic compounds obtained by steam distillation and cold expression or cold pressing of certified organic plant materials (including the stems, branches, fruits, fruit rinds, flowers, seeds, roots, bark, needles, leaves and any other part of the plant) in a way that preserves the essential oil in a form that is as close to nature as the extraction process will permit. The use of all natural, pure, organic essential oils is preferred. An alternative to the preferred could include the use of standardized or synthetically produced essential oils and/or their chemically active components that are known to reduce, retard or eradicate mold, such as cinnamaldehyde, eugenol, citral, geraniol, carvacrol, and thymol.

### DETAILED DESCRIPTION OF THE PREFERRED METHODOLOGIES

The present invention concerns a novel process for the remediation of mold from dwellings, offices, and other enclosed buildings where the growth of mold has created unsafe or intolerable conditions for humans, through the use of essential oils. Because over the counter products may not be truly pure therapeutic grade essential oil, standards have been created to differentiate the essential oils that are merely diluted or synthetic substitutes. AFNOR (Association Francaise de Normalization) and ISO (International Standards Organization) standards are often found on labels that have been independently tested and found to meet a standardized minimum profile set by the essential oils industry. The AFNOR/ISO standards attest to a high degree of certainty as to the purity of oils, because the AFNOR-adopted quantities of components correspond to those found in natural products. Hence, according to AFNOR authorities, if an essential oil fits AFNOR/ISO standards, it is generally considered to have come from a natural source.

The present inventors have discovered that continuously diffusing essential oils into an infected home or building can rapidly, safely, and thoroughly eliminate many mold infestations without harmful chemicals or unpleasant odors. Further, preventative measures including diffusing oils can prevent the onset of mold infestation and protect the value of the property in an inexpensive and safe manner.

Where there is no known mold problem and no evidence of mold problems are present, mold growth can be prevented by diffusing essential oils for fifteen to thirty minutes every three to four hours at least once each week in each room, or by performing a short, intensified diffusion of essential oils once a week for eight hours in each room, followed by monthly maintenance checks. Diffusing essential oils in living spaces and offices not only prevents the growth of mold and the potential costs associated with mold remediation, but may have profound health benefits as well by reducing incidents of flu, colds, bronchitis, sinusitis, and chronic allergies.

Both the preventative and remediation steps involve the diffusion of essential oils into the affected areas. The essential oils preferably comprises of a blend of at least five 100% pure, organically produced, therapeutic-grade essential oils that are approved by the FDA as food supplements. The preferred combination of essential oils comprises extracts of Clove, Rosemary, Cinnamon, Lemon, and Eucalyptus radiata (referred to hereafter as 5-part essential oil). Such mixture of oils can be obtained, for example, from Young Living Essential Oils, of Lehi, Utah, sold under the trademark Thieves™ essential oils. The other oils that may have preventative benefits include Lemongrass, Oregano, Eucalyptus, Cassia, Cinnamon Bark, Citronella, Thyme, Marjoram, and Melaleuca oils, as well as a variety of other individual or single essential oils and oil blends.

To achieve the necessary diffusion properties, the diffusion unit utilized is preferably a cold-air diffuser capable of producing a fine, micro-mist dispersion of essential oils. One diffuser can be used for about 1,000 square feet of floor space when the pump is rated at 3.5 to 4.5 watts. In general, diffusion to prevent mold in a home or office that is 1,500 square feet or smaller requires one 15 ml bottle of essential oils over the course of a month. The well of the diffuser is filled and the diffuser may be placed on a timer if desired.

A large variety of diffusers are available for dispersing essential oils. They should be sturdy, easily cleaned, and very effective at delivering a high volume of essential oil in a micro-fine mist that will permeate an entire room in a matter of minutes. A preferred diffuser is a cold-air diffuser that utilizes a non-reactive metal well to hold the essential oil, a glass nebulizer to disperse the oil in a micro-fine mist, and an air pump that forces the oil into and through the nebulizer. The size of the pump used in most of the case studies was 115 volts, 60 hrz, 3.5 watts. This type of diffuser will provide a concentrated micro-mist of essential oil at a high dispersion rate and will permeate the air in a space in a matter of minutes. The volumetric throughput of such a unit can be approximately one liter of air per minute and about 0.5 ml of oil per hour.

Diffusers that utilize heat or water should be avoided. The diffusers that use heat destroy the most fragile organic compounds and also pose a fire hazard. Diffusers that use water provide moisture for mold growth. Diffusers that use water and diffusers that use heat fail to disperse adequate amounts of the essential oil to be effective for mold prevention or remediation. Diffusers that do not have an electric air pump to force cold air through the well of oil may not disperse adequate amounts of the essential oil to be effective for mold prevention or remediation, and are also not preferred.

### Step 1. Sampling

Toxic mold must be dealt with properly to avoid unnecessary exposure and potential health impacts. The only way to know if a toxic-mold problem is present is by sampling. Reasons for sampling include: (1). If it is assumed any mold is benign and attempts are used to remove it using rubber gloves, a sponge, and bleach water, and a toxic mold like Stachybotrys chartarum, Chaetomium, or certain species of Aspergillus or Cladosporium, are present, serious health problems can result. (2). Tens of thousands of mold spores per cubic meter can be present and not be seen, yet they are detected by appropriate air sampling. Without proper sampling, the nature and severity of the problem cannot be known with any degree of certainty. (3). Even if all visible molds are removed, there may still be a hidden problem that could be identified with proper sampling. (4). A cleanup method that may be safe for some mold infestations can be disastrous for others. (5) Without adequate sampling, every remediation effort should be carried out assuming the mold is highly toxic, which is a very costly approach.

Several methods are utilized in mold sampling, including: Impactor Air Sampling, Viable Air Samples; Tape Lift Samples; Bulk Samples; and Wall Probes. One or more of these methods will be appropriate to different site-specific circumstances. New sampling methods are being devised every year for specific conditions and needs. For example, if a high level of certainty is necessary for legal proceedings, species identification using DNA sequencing may be appropriate.

Impactor air sampling consists of setting up an air pump and pulling air though a specially manufactured device that traps the spores that happen to be in the air. Outdoor and indoor samples are taken in order to determine which mold species are growing inside. The pump must also be accurately calibrated so that one knows exactly how much air it moves per unit of time in order for the laboratory to provide accurate results. The time and pumping rate are recorded so that the exact volume of air pulled through the trap can be determined. In the lab, a microbiologist determines the total number of spores of each of the mold species per cubic meter of air.

Advantages of this method of sampling include: (1). If the pump is operated for a sufficient length of time and a sufficient volume of air is sampled, the results are representative of an extended space, such as a room or office. (2). If the results are compared with a spore-trap sample of the ambient air outside, the species of mold suspected of growing inside the space can be identified. Disadvantages include: (1). Results can vary due to any of a number of activities disturbing the mold and spores in the test space, and to a minor degree, the variability of laboratory analytical methods. (2). Some mold species cannot be specifically identified using this method. For example, Aspergillus and Penicillium spores are indistinguishable in this method and are identified in the count provided on the lab's report as Penicillium/Aspergillus. (3). This type of sample provides information on spore counts but does not distinguish between dead and living (viable) spores.

The second method of sampling, Viable air samples, uses an air pump to collect spores on an agar plate in a special type of impactor. The agar provides a base or substrate on which the mold spores can grow. Advantages of this method include: (1). This type of air sample allows the lab to identify living (viable) mold spores that can produce new colonies of mold. (2). The test enables one to make a more reliable determination of which mold species are growing inside the building. (3). Better identification of individual mold species is possible. (4). Differentiation between Aspergillus and Penicillium is possible. Disadvantages include: (1). It takes longer to get results. (2). Viable sample analysis is usually more expensive than nonviable.

For Tape Lift Samples, direct examination by a microbiologist or mycologist will determine the specific mold species growing on the tape. In principle, this method is very simple: A piece of clear tape is placed on the mold, then lifted from the surface carefully and placed in a sealable clear plastic bag and shipped to a microbiology lab for analysis. The advantage of this method is its simplicity. The disadvantages are: (1). It only yields information about the one small spot where the tape was pressed. (2). The structure of the mold may be crushed or smeared, making identification of species difficult. (3). The mold growth may be masked by lint, particulate matter, and other debris stuck to the tape.

In a bulk sample test, a piece of material (typically about one inch square) cut from the surface upon which mold growth is visible is collected, sealed in a plastic bag, and shipped to the laboratory for analysis. Advantages of this type of sampling include: (1) Bulk samples are better than tape lift samples for positive identification of mold species because growth structure is not damaged. (2) Like viable air samples, the sample can be cultured to determine which mold species are alive and growing on the material. Disadvantages include: (1) Cutting damages the material, eliminating the possibility of cleaning it and leaving it in place. (2) Like the tape-lift method, this type of sample only yields information about the one small spot from which it was collected.

With Wall Probes, a small hole about one-fourth inch in diameter is drilled through the wall behind which a mold infestation is suspected. A probe is inserted with an airtight seal, and air is drawn from inside the wall cavity into a spore trap or impactor. Advantages of this methodology include: (1). A wall probe sample allows you to determine whether mold growth exists inside a wall cavity without the time and materials expenses involved in removing a section of the wall. (2). This method avoids disturbing mold spores and is, therefore, safer than more invasive procedures. (3). The hole can be easily repaired with spackling or putty and paint. Disadvantages include: (1). It may be difficult to determine exactly where to drill. (2). Many holes may have to be drilled in structures with solid studs and sills that create barriers to air movement within the wall.

Sampling methods are determined by the indicators found by inspection. The process of selecting the proper sampling techniques is illustrated in Table One.

| | Sampling Methods | | | | |
|---|---|---|---|---|---|
| | | | Viable | | Wall |
| Mold Indicator | Spore Trap | Tape | Bulk* | Viable Air* | Probe^{†} |
| Mold on Wall | X | X | | X | X |
| Mold on Carpet | X | X | X | X | |
| Mold in Crawl | X | | | X | |
| Space | | | | | |
| Mold in HVAC | X | X | | X | |
| System | | | | | |
| Water Damage | X | X | X | X | X |
| Dampness | X | | | X | X |
| Odor | X | | | X | |
| Health Problems | X | | | X | |

*Bulk sampling is not always possible, since a piece of material must be removed, but at least one viable bulk or air sample should be collected to help design an effective protocol.
^{†}Spore trap samples should be obtained through wall-probe tubes if there is reason to suspect that mold is growing inside wall cavities.

One or more outdoor samples are collected first to establish an ambient baseline with which to compare the results of indoor sampling. One outdoor spore trap air sample should be collected at least 50 feet from entry/exit doors, and one from each HVAC intake.

The number of samples to be taken depends upon the sizes and number of rooms in the building. At least one sample should be collected on each floor of the building, with basements and attics included as floors. On a given floor, at least one sample should be collected for each room up to 1000 square feet of floor space or 8,000 cubic feet of space if ceilings are higher than eight feet. OSHA has guidelines and training pertaining to the collection of biohazards such as mold, and the collection is properly carried out by qualified professionals with a minimum of 40 hours of OSHA training, whereupon the collected specimens are shipped to a qualified microbiology laboratory for analysis.

When the laboratory reports are received, the first step is to determine which mold species are present and growing inside the building, so that the diffusing/cleaning protocol can be designed to fit the circumstances at hand. Comparison of the spore concentrations in the indoor samples with concentrations found in the outdoor samples can be used to determine which species are growing inside the building. Under normal circumstances, e.g., with an air-conditioned building with doors and windows closed, calm, dry weather conditions, and little activity indoors or out that would stir up mold spores, the spores of species found outside, may also be found inside at lower levels. It follows that, under these “normal” circumstances, all species with spore concentrations equal to, or higher than, outside levels, are probably growing somewhere in the building. If, on the other hand, the building has no air conditioning, the doors and windows are open, there is high traffic activity in and out, and/or if windy conditions are causing fluctuations in outdoor spore concentrations, indoor concentrations that are equal to, or only slightly higher than outdoor levels, are not reliable indicators. Even under these “adverse” conditions, however, if the indoor concentration of a given species is several times the outdoor level, this fact strongly suggests that a source of the mold is growing inside the building.

In cases where the indoor/outdoor mold spore concentration comparison is inconclusive, the results from other types of sampling can resolve the question. For example: if tape (sometimes called tape-lift) samples or bulk samples from inside the building show Cladosporium growth, Cladosporium is growing inside the building, even if the indoor concentration of Cladosporium spores is less than the outdoor concentration.

### Step 2. Diffuse:

The tables below are preferred steps to design an objective, site-specific treatment protocol based on quantitative sampling results. If the mold spores growing inside are also found in outdoor samples, diffuse with the 5-part essential oils and clean visible mold and stain from exposed surfaces with a household cleaner having 5-part essential oils therein according to Table Two. If one or more mold species are found inside, but not outside, diffuse with 5-part essential oils and clean visible mold and stain from exposed surfaces with a household cleaner having 5-part essential oils therein according to Table Three. If the species found inside but not outside include any of the following: Stachybotrys, Aspergillus, Penicillium, Acremonium, Cladosporium, Alternaria, Chaetomium, Torula, Memnoniella, and Curvularia, diffuse 5-part essential oils and clean with a household cleaner having 5-part essential oils therein according to Table Four.

TABLE TWO

| | Indoor to | Colony- | Hours of | | |
|---|---|---|---|---|---|
| | Outdoor | Forming | Continuous | Essential | |
| | Ratio (R)^{a} | Units^{b} | Diffusion | Oils | Cleaner |
| For Mold | 1 ≦ R < 2 | 1-500 | 24 | 5-part | Household |
| Species | | | | essential oils | cleaner + 5- |
| Found Both | | | | | part essential |
| Inside and | | | | | oils |
| Outside | | | | | undiluted |
| | 2 ≦ R < 10 | 500-1000 | 36 | 5-part | Household |
| | | | | essential oils | cleaner + 5- |
| | | | | | part essential |
| | | | | | oils |
| | | | | | undiluted |
| | 10 ≦ R | >1000 | 48 | 5-part | Household |
| | | | | essential oils + Ci | cleaner + 5- |
| | | | | | part essential |
| | | | | | oils |
| | | | | | undiluted + additional |
| | | | | | essential oils |

^{a}Spore Trap Samples
^{b}Viable Samples

If the results of a and b do not agree, use the more aggressive level of diffusing and cleaning indicated.
Where the cleaner is mixed with the essential oils, add approximately one measure of 5-part essential oils to three measures of household cleaner including 5-part essential oils.
Ci=Cinnamon essential oil. Add one measure Ci. to four measures 5-part essential oils.

TABLE THREE

| | Spore Trap | Colony- | Hours of | | |
|---|---|---|---|---|---|
| | Concentration | Forming | Continuous | Essential | |
| | in spores/m³ | Units | Diffusion | Oils | Cleaner |
| For Mold | 1-10 | 1-500 | 24 | 5-part | Household cleaner + 5- |
| Species | | | | essential | part essential oils |
| Found | | | | oils | undiluted |
| Inside But | 10-100 | 500-1000 | 36 | 5-part | Household cleaner + 5- |
| Not | | | | essential | part essential oils |
| Outside* | | | | oils | undiluted |
| | >100 | >1000 | 48 | 5-part | Household cleaner + 5- |
| | | | | essential | part essential oils |
| | | | | oils + Ci | undiluted + additional |
| | | | | | essential oils |

*Unless they are among the list addressed in Table Four.

Where the cleaner is mixed with the essential oils, add approximately one measure of 5-part essential oils to three measures of household cleaner including 5-part essential oils.
Ci=Cinnamon essential oil. Add one measure Ci. to four measures 5-part essential oils.

TABLE FOUR

| | Spores/m³ | CFUs | Hours | Oils | Cleaner |
|---|---|---|---|---|---|
| For Mold | 1-10 | 1-500 | 24 | 5-part | Household |
| Species | | | | essential oil + CiLgO | cleaner + 5- |
| From the | | | | | part essential |
| List Below* | | | | | oils undiluted + additional |
| | | | | | essential oils |
| | 10-100 | 500-1000 | 48 | 5-part | Household |
| | | | | essential oil + CiLgO | cleaner + 5- |
| | | | | | part essential |
| | | | | | oils undiluted + additional |
| | | | | | essential oils |
| | >100 | >1000 | 72 | 5-part | Household |
| | | | | essential oil + CiLgO | cleaner + 5- |
| | | | | | part essential |
| | | | | | oils undiluted + additional |
| | | | | | essential oils |

*Stachybotrys, Aspergillus/Penicillium, Acremonium, Cladosporium, Alternaria, Chaetomium Memnoniella, and Curvularia.
CiLgO = Cinnamon, Lemongrass or Oregano essential oil is added to the 5-part essential oil.

If mold growth is found inside an HVAC system, then, in addition to the protocols indicated by these tables, remove the panel in front of the “A” coil and spray all visible mold and stains with the cleaner mixture specified in the appropriate table, using a paint sprayer with a non-corrosive metal nozzle. This is preferably accomplished using a new commercially available paint sprayer that has not been used to spray paint or chemicals other than the cleaner or mixtures specified above. At least one liter of the cleaner mixture per 2000 square feet of space served by the HVAC system is sprayed into the HVAC system with the fan running.

### Step 3. Repair Leaks:

Preventing mold infestations in existing buildings primarily involves controlling moisture on surfaces that can support mold growth. This can be achieved using one or more of the following steps: (1). Fix water leaks and prevent excess moisture; (2). Control indoor humidity; (3). Conduct regular cleaning of and inspection of areas where moisture may create mold problems; (4). Make sure there is proper ventilation; (5). Keep mold-infested materials out of the building; and (6). Reduce and/or eliminate mold spores in indoor air by diffusing essential oils regularly.

The most common reason that molds start growing inside a building, causing health problems for the inhabitants, is excess moisture. Excess moisture results from high levels of indoor humidity causing condensation on exposed metal surfaces, water leaks, and other forms of water intrusion into the home or building. Moisture from condensation can penetrate building materials such as sheetrock, wallpaper, and wood, and create an ideal environment for mold spores to start colonies. Covering metal surfaces with insulation helps to prevent condensation.

Condensation on or around a window is a sign of inadequate insulation and/or excessive moisture. Mold has been observed to grow on window sills and even on glass panes and aluminum frames. This source of excess moisture can be eliminated by installing storm windows and/or using a sealant around the window to make sure that it is indeed airtight.

It is important to keep relative-humidity levels in all parts of a building below 55% if possible to control mold growth. Toxic molds, bacteria, and dust mites thrive when the relative humidity is at or above this level. If humidity is too high, installation of relative-humidity sensors, called hygrometers or moisture meters, can help alleviate the problem. Each room or enclosed space can be monitored separately, especially bathrooms, basements, crawl spaces, and cellars. It is advantageous to also monitor humidity levels in ductwork, especially near the air-handling equipment and filter locations and inside or between walls. It is not unusual to find the source of a mold problem in HVAC ductwork.

### Step 4. Clean Thoroughly

Use the household cleaner containing undiluted essential oils to clean all areas of visible mold. One such cleaner is available from Young Living Essential Oils, of Lehi, Utah, sold under the trademark Thieves™ Household Cleaner. The Thieves™ cleaner is a household cleaner that also includes the Thieves™ essential oils blend therein. If necessary, as indicated by the tables above, it is preferable to add an additional 15 milliliter bottle of the essential oils blend to the household cleaner that already has the essential oils premixed therein. The household cleaner with essential oils may be sprayed in crawl spaces and on metal ductwork. It may also be used to clean tiles, sheetrock, and other porous materials that have small areas of visible mold.

### Step 5. If Step 2 Sampling Results Identify Toxic Mold

Begin intensive diffusion of essential oils immediately and continue non-stop diffusing for 24 to 72 hours, depending on the protocol set forth herein. It is important to remove and properly dispose of water-damaged and mold-infested materials having the toxic mold growing thereon.

### Step 6. Remove, Repair, and Replace Water-Damaged and Mold-Infested Materials.

Removal of mold-infested materials involves tearing out moldy porous materials, such as wallpaper, sheetrock, and plywood. Such activities disturb mold, and mold spores become airborne, increasing the risk of exposure. All materials are properly sealed in plastic bags before disposal to ensure the safety of others.

### Step 7. Re-Sample

Collecting samples again following all remediation efforts is necessary to be sure the problem has been solved. Simple visual inspection is inadequate because mold spores are not visible to the naked eye. If the protocol has been followed, and mold spores are still present, especially at elevated levels, then the source or an additional source of the mold may not have been discovered. If re-sampling reveals one or more species with elevated spore concentrations, molds are still growing somewhere in the building.

### Step 8: Repeat Steps 1 Through 7, If Necessary

Among all the opportunities the present inventors have experienced, only one test case required repeating Steps 1 through 7. The reason was not because the first application had not worked; but rather a different source of mold spores, a previously undetected growth of Stachybotrys chartarum colonies, had not been discovered during the initial sampling. This step is included in the protocol expressly because this could happen in any building where there are multiple locations with conditions conducive to mold growth.

### Step 9: Refinish Affected Areas

This is the final step in remediating mold. This step is necessary only in cases where mold-infested and/or water-damaged materials such as wallpaper, sheetrock, wood paneling, walls, ceilings, wood framing or other structural materials have been removed. This step should not be undertaken until sampling results have verified a substantial or complete reduction in mold spores of concern.

### Step 10: Diffuse Regularly.

Continuing to diffuse essential oils fifteen minutes every three to four hours at least three times a week is recommended as a preventative measure. Conditions conducive to mold infestation can occur in any building wherever excess moisture appears. As a building ages small leaks may develop in roofs, around windows, in piping, or in other locations, and these may go unnoticed until it is too late to prevent mold growth.

The present inventor derived a method for measuring the efficiency of diffusing and cleaning with the essential oils. The Spore Removal Efficiency of a treatment was defined as the percentage of available mold eliminated by the treatment.

### Calculation of Spore Removal Efficiency (SRE)

The SRE is equal to available spores per unit volume of air during treatment, minus spores per unit volume of air remaining after treatment, divided by available spores per unit volume during treatment, multiplied by 100 to convert the decimal result into a percentage. When a given species is found in samples taken inside the space being treated but not found in outdoor samples, the available number of spores per unit volume (cubic meter of air) is given by the lab report for the indoor sample collected immediately before treatment started, and calculation of the SRE is straight forward.

### Equation:

$$SRE = \frac{Cib - Cia}{Cib} \times 100$$

C=Concentration of mold spores per cubic meter of air
i=Inside
b=Before treatment
a=After treatment

If, however, the species is found in both the inside and the outdoor samples, the number of available spores per cubic meter of air during treatment is affected by the amount of exchange of air between the indoor environment and the ambient air outside.

$$SRE = \frac{Cib + Eavg - Cia}{Cib + Eavg}$$

where Eavg=the average exchange during the treatment in mold spores per cubic meter.

This exchange can be positive or negative for a given species. It is also affected by the size, weight, and other physical features of the spores of each mold species. The number of available spores per unit volume was determined for each species and for the total spores for all species combined in each case study, to avoid to the extent possible over- or under-estimating the SREs. Calculation of the SRE for each case affords a relative measure of the effectiveness of this, or any other protocol.

The following examples are case studies of the process described above, with SREs calculated. Case studies No. 1, A Real Estate Office, which was a converted Victorian home, No. 2, A Private Bootheel-Area Residence, that was being remodeled, and No. 3, An Immaculate Residence, that happened to have mold in the HVAC system, are good examples.

Case Study No. 4, A Hospital and NICU, where the solution to one problem uncovered another one, is a good example of the value of the SRE calculation and the importance of the indoor/outdoor air exchange in cases where the spore count is low. In this case, the air flow into and out of the neo-natal intensive care unit (NICU) was measured carefully in order to aid in the detective work to discover the source of Stachybotrys mold spores.

Case Study #1 (A Real Estate Office)

This is the case of a seventy-five-year-old Victorian style home in Texas County, Missouri, that had been converted to a real estate office. Agents working in the offices complained of headaches, coughing, and general respiratory irritation. Inspection of the building revealed numerous leaks, especially in one second-floor room and around a chimney that had been out of use since the heating had been converted from wood-burning stoves to a central air and heat system. There was also an earth-contact basement with a sump pump. The basement was closed off from the remainder of the building, and the client did not permit entry or sampling in the basement. The earthen floor in the basement was reported to be always damp, with water in the sump.

Outdoor and indoor air samples were collected, and tape-lift samples were taken from the most prominent colonies of visible mold. This building had a number of small rooms upstairs, and several small rooms on the main floor off a larger living room and a centrally located stairwell. The leaks were repaired, and the 5-part essential oils mixture was diffused continuously for 24 hours in a central location on the ground floor of the building. Approximately 15 ml of the 5-part essential oils mixture was dispersed in the space.

The agents working in the offices noticed almost immediate relief of headaches and respiratory symptoms following the diffusion of the essential oils. Follow-up samples were collected seventeen days later and again five months later. There was no diffusion or mold cleaning, no removal or remediation during the interim periods.

Observations and Conclusions

Ten species of mold, including six known toxic species, were found growing in the building. Spore-removal efficiency was 100% for Stachybotrys chartarum, Chaetomium, Scopulariopsis, and Trichoderma, with an overall SRE of 99.3%. Long-term effects of diffusing on spore counts and health symptoms are also demonstrated by this case study. Even though there was a slight rebound after five months, spore counts were still far below the levels detected in the samples collected before treatment with the 5-part essential oils. The results of the tests are shown below in Table 6.

TABLE 6
CASE STUDY NO. 6

| Mold Species | Cib | Cob | Cib/Cob | Cia1 | Cia2 | Coa2 | Cia/Cob | SRE |
|---|---|---|---|---|---|---|---|---|
| Alternaria | 433 | 693 | 0.62 | 7 | 7 | 0 | NA | 99.1% |
| Ascospores | 0 | 2940 | 0.00 | 0 | 13 | 573 | 0.02 | 99.3% |
| Basidiospores | 267 | 380 | 0.70 | 7 | 93 | 20773 | 0.004 | 99.1% |
| Bipolaris/ | 0 | 13 | 0.00 | 0 | 0 | 0 | NA | 100.0% |
| Drechslera | | | | | | | | |
| [symbol] | 160 | 0 | GIO | 0 | 0 | 0 | NA | 100.0% |
| [symbol] | 6467 | 3327 | 1.94 | 27 | 73 | 633 | 0.12 | 99.1% |
| [symbol] | 80 | 7 | 11.43 | 0 | 7 | 0 | NA | 91.6% |
| Epicoccum | 220 | 533 | 0.41 | 0 | 13 | 27 | 0.48 | 97.4% |
| Fusarium | 87 | 160 | 0.54 | 0 | 0 | 0 | NA | 100.0% |
| Other Colorless | 0 | 7 | 0.00 | 0 | 0 | 13 | 0.00 | 100.0% |
| Other Brown | 20 | 0 | GIO | 20 | 7 | 13 | 0.54 | 73.6% |
| [symbol] | 34147 | 440 | 77.61 | 33 | 220 | 240 | 0.92 | 99.4% |
| [symbol] | | | | | | | | |
| Rusts | 0 | 7 | 0.00 | 0 | 0 | 0 | NA | 100.0% |
| Smuts, etc. | 587 | 87 | 6.75 | 0 | 0 | 13 | 0.00 | 100.0% |
| [symbol] | 13 | 7 | 1.86 | 7 | 0 | 0 | NA | 100.0% |
| Torula | 13 | 40 | 0.33 | 0 | 0 | 0 | NA | 100.0% |
| Fusicladium | 0 | 7 | 0.00 | 0 | 0 | 0 | NA | 100.0% |
| [symbol] | 13 | 0 | GIO | 0 | 0 | 0 | NA | 100.0% |
| [symbol] | 120 | 40 | 3.00 | 0 | 0 | 0 | NA | 100.0% |
| Spegazzinia | 0 | 7 | 0.00 | 0 | 0 | 0 | NA | 100.0% |
| [symbol] | 440 | 0 | GIO | 0 | 0 | 0 | NA | 100.0% |
| Total | 43067 | 8695 | 4.95 | 101 | 433 | 22285 | 0.02 | 99.3% |

C = Concentration (Mold Spores per cubic meter) i = inside, b = before, o = outside, a = after
SRE = Spore Removal Efficiency
Bold indicates growth inside the building
Cib & Cob were sampled on Jul. 22, 2006;
Cia1 on Aug. 8, 2006;
Cia2 & Coa2 on Jan. 4, 2007
NA = Not Appropriate
GIO = Growing inside Only

Case Study #2 (A Private Residence)

This house had a minor roof leak around a rock fireplace. It also had a basement, which is unusual in this area due to a high water table that ranges seasonally from four to eight feet below land surface. The basement was equipped with a sump pump to handle seepage, and the sump always had standing water in it, providing a never-ending source of excess moisture. This house also had two earth-contact crawl spaces.

While renovating the house, workers found mold growing on the ceiling around the fireplace, under carpets on the main floor, and heavy growth on the outside of HVAC ducts in the basement. The owner contacted the present inventors to identify the mold species and advise her regarding cleanup and removal of the mold. Her husband and two family pets had died of cancer during the past year, and renovations at the home were being undertaken to allow her daughter's family to move in to the home. She was an outdoor type of person, who spent little time inside the house, and had not noticed any adverse health-related symptoms that she attributed to mold. Her husband and pets had spent almost all of their time in the house during the past year, due to illness, and the woman wondered if the mold might have contributed to their untimely deaths.

Observations and Conclusions

Air and tape-lift samples showed at least eight species of mold growing in the house, including Stachybotrys chartarum, Aspergillus, Chaetomium, and Epicoccum. High levels of Graphium were found growing on and under the carpet. All of these species are toxic, or potentially toxic, and have been linked by scientific research to serious diseases including cancers.

Because the total floor space of the house was more than 2000 sq. ft., the 5-part essential oils mixture was diffused continuously in a central location on the main floor and also in the basement for 48 hours, dispersing four 15 ml bottles of the blend. Visible mold and the crawl spaces were sprayed with the cleaner containing the 5-part essential oil mixture. The results are displayed in the table and graphs that follow.

The treatment used at this location included diffusing the 5-part essential oil mixture continuously for 48 hours and spraying visible mold and crawl spaces with the household cleaner containing the 5-part essential oil mixture. The treatment proved very effective. The SREs for seven species and groups of molds were 100%, and the overall SRE was 99.1%.

TABLE 10
CASE STUDY NO. 10

| Mold | | | | | | | |
|---|---|---|---|---|---|---|---|
| Species | Cib | Cob | Cib/Cob | Cia | Coa | Cia/Coa | SRE |
| [symbol] | 27 | 0 | GIO | 0 | 13 | 0.00 | 100.0% |
| Ascospores | 180 | 387 | 0.47 | 0 | 47 | 0.00 | 100.0% |
| Basidio- | 87 | 2553 | 0.034 | 33 | 847 | 0.039 | 98.2% |
| spores | | | | | | | |
| [symbol] | 293 | 0 | GIO | 0 | 7 | 0.00 | 100.0% |
| Clado- | 420 | 433 | 0.97 | 80 | 120 | 0.667 | 88.5% |
| sporium | | | | | | | |
| [symbol] | 60 | 0 | GIO | 0 | 7 | 0.00 | 100.0% |
| Other | 53 | 33 | 1.61 | 0 | 0 | NA | 100.0% |
| Colorless | | | | | | | |
| Other Brown | 27 | 0 | GIO | 20 | 7 | 2.86 | 34.4% |
| [symbol] | 15593 | 73 | 213.60 | 40 | 107 | 0.374 | 99.7% |
| [symbol] | | | | | | | |
| Smuts, etc. | 20 | 0 | GIO | 7 | 13 | 0.538 | 73.6% |
| [symbol] | 13 | 0 | GIO | 0 | 0 | NA | 100.0% |
| [symbol] | 133 | 0 | GIO | 0 | 0 | NA | 100.0% |
| Total | 16906 | 3479 | 4.86 | 180 | 1168 | 0.15 | 99.1% |

C = Concentration (Mold Spores per cubic meter) i = inside, b = before, o = outside, a = after
SRE = Spore Removal Efficiency
Bold Indicates growth inside the building
NA = Not Appropriate
GIO = Growing inside Only

Case Study #3 (A Private Residence)

Upon entering this beautiful, spacious home, it would be hard for anyone to imagine that there could be a mold problem. It was immaculate, with no visible signs of mold anywhere. The couple who lived in the home was still in the prime of their lives. The wife was a business owner who had retired early, and the husband had suffered a debilitating work-related back injury. Consequently, both spent considerable time in the house. They had had an addition built onto their home and had the HVAC system reconfigured during the renovations. Not long after that project had been completed, both husband and wife began suffering from chronic headaches, dizziness, nausea, and chronic sinus problems which they attributed to stress and weather conditions.

On a routine maintenance visit, their HVAC repairman found black mold growing on a panel covering the A-coil in the central air conditioning unit. In spite of the fact that there was no obvious visible mold, this case study had higher mold-spore concentrations for two toxic mold species and higher total mold spores than any other case study, even those where an abundance of visible black mold was found. This underlines the importance of sampling.

A bulk sample taken from the HVAC panel proved to be a very heavy growth of Cladosporium, and a spore-trap air sample collected near outlet vents on the main floor, taken with the HVAC system running, revealed at least five species of mold growing inside the house, including Stachybotrys, Curvularia, and Penicillium/Aspergillus. Concentrations of Penicillium/Aspergillus and Cladosporium spores were very high at 38,773 and 19,573 spores per cubic meter, respectively, with total mold spores more than three hundred times the total in the outdoor sample.

The mold-infested HVAC panel was sprayed with the household cleaner containing the 5-part essential oils mixture and one additional 15 ml bottle of the 5-part essential oil mixture was added. Two diffusers on the main floor dispersed the 5-part essential oil mixture continuously over a period of more than 72 hours, using approximately 60 ml of the essential oil blend. Post-treatment samples were collected six days later. The post-treatment indoor air sample was collected in the same location, with the HVAC system operating, as had been done when collecting the original indoor sample.

Observations and Conclusions

The results of diffusing and spraying in this case study were excellent. Twelve mold species and groups were found in the before-treatment air samples. The SREs for eight of these were 100%, with an overall SRE of 99.8%. See Table 11 for indoor/outdoor ratios and SRE values. Another set of post-treatment samples taken about one and one-half months later showed the indoor spore levels remained far below outdoor levels. The occupants of the home reported that their symptoms were markedly reduced and that they very much enjoyed the smell of the essential oil blend. The couple in this case noticed an improvement in their symptoms within 24 hours of the time the treatment commenced. They remained inside the home throughout the treatment period, and they have decided to continue diffusing the oil blend at least once a week as a preventative measure to protect their home and their property.

TABLE 11
CASE STUDY NO. 11

| Mold | | | | | | | |
|---|---|---|---|---|---|---|---|
| Species | Cib | Cob | Cib/Cob | Cia | Coa | Cia/Coa | SRE |
| Ascospores | 0 | 7 | 0.00 | 0 | 87 | 0.00 | 100.0% |
| Basidio- | 40 | 107 | 0.37 | 44 | 2747 | 0.02 | 97.0% |
| spores | | | | | | | |
| [symbol] | 19573 | 140 | 139.81 | 22 | 713 | 0.03 | 99.9% |
| [symbol] | 7 | 0 | GIO | 0 | 0 | NA | 100.0% |
| Epicoccum | 7 | 13 | 0.54 | 0 | 13 | 0.00 | 100.0% |
| Fusarium | 0 | 0 | NA | 0 | 7 | 0.00 | 100.0% |
| Other | 0 | 93 | 0.00 | 0 | 93 | 0.00 | 100.0% |
| Colorless | | | | | | | |
| Other Brown | 20 | 147 | 0.14 | 0 | 147 | 0.00 | 100.0% |
| [symbol] | 38773 | 273 | 142.03 | 76 | 140 | 0.54 | 99.8% |
| [symbol] | | | | | | | |
| Smuts, etc. | 47 | 33 | 1.42 | 4 | 27 | 0.15 | 94.8% |
| [symbol] | 7 | 0 | GIO | 0 | 0 | NA | 100.0% |
| Torula | 0 | 20 | 0.00 | 0 | 20 | 0.00 | 100.0% |
| Total | 58474 | 833 | 70.20 | 146 | 3994 | 0.04 | 99.8% |

C = Concentration (Mold Spores per cubic meter) i = inside, b = before, o = outside, a = after
SRE = Spore Removal Efficiency
Bold Indicates growth inside the building
NA = Not Appropriate
GIO = Growing inside Only

Case Study #4 (A Hospital and NICU)

This case study took place in a hospital. Hospital administrators requested that samples be taken in several areas, including operating rooms, intensive care units, x-ray rooms, break rooms, and offices, specifically those frequented by a nurse who was having allergic reactions to something in the hospital. The nurse would break out in a rash fifteen or twenty minutes after arriving at work. Her doctor was not able to determine the cause and suggested that it might be a reaction to mold. Extensive sampling found mold spores in these locations but not of sufficient concentrations or species types to cause such reactions. The cause was finally determined to be her extreme sensitivity to toxic chemicals in a solvent that was used to clean the operating rooms.

At a later time, a few Stachybotrys spores were found in an air sample from a doctor's station located adjacent to a neo-natal intensive-care unit (NICU). This concerned the hospital's doctors and administrators for several reasons: First, Stachybotrys spores are suspected of being dangerous to persons with compromised or undeveloped immune systems, especially newborn babies. Second, Stachybotrys spores are rarely, if ever, found in air samples unless there is growth nearby, because Stachybotrys spores are heavy, sticky, and not easily airborne. Third, this was a clean-room area, in which air pressure and oxygen content were controlled, and anyone entering had to wear a breathing mask and disposable protective clothing, and wash his/her hands with disinfecting soap upon entering the area. Furthermore, the air-handling system was protected with HEPA filters that should have stopped mold spores from entering the building through the air exchange of the HVAC system.

After some detective work, including taking air samples from the overhead space above the ceiling tiles and from the HVAC return and measuring the air flow into and out of the NICU, the present inventors found that the spores were coming into the area from the overhead space, due to an ineffective seal put in place during remodeling of the NICU room. The spores were then traced to a stairwell. A leak had developed where the flat roof abutted with the stair well, allowing water to run down the inside of the stairwell wall. When the drywall was removed from the stairwell wall, large areas of black mold were exposed. The water-damaged and mold-infested drywall was removed, and the 5-part essential oils mixture was diffused continuously for 24 hours in the stairwell and in the NICU.

Observations and Conclusions

The analytical data represented in the table below are for the area just outside the door to the NICU where the problem was traced to the overhead space and stairwell. The overall spore removal efficiency of the essential oils protocol utilized at this hospital was 99.3%. Some intermediate samples were taken, after diffusing in the NICU, but before the stairwell and overhead were treated and resealed. Those results showed a high level of exchange between the indoor and outside air. The after sample used in the graph and table is representative of the conditions after the project was completed. While mold spores were still found in every room tested, the levels were extremely low (10 to 60 total mold spores per cubic meter), and there were no Stachybotrys spores.

The indoor/outdoor ratios in the before sample are almost all less than one for all but three species, and they had very low counts. Under most circumstances, this would not indicate a cause for concern over potential mold exposure. However, under these circumstances, in a hospital where patients typically have weak immune systems, finding even one Stachybotrys spore is cause for concern. The overall efficiency of the essential oils protocol again proved to be very good at 99.3% removal. The three spore types with SRE values less than 100% are light, easily air-borne spores that are abundant outside and are often carried inside on clothes and hair. The results are shown below in the Table below.

TABLE 8
Case Study NO. 8

| Mold Species | Cib | Cob | Cib/Cob | Cia | Coa | Cia/Coa | SRE |
|---|---|---|---|---|---|---|---|
| Ascospores | 0 | 187 | 0.00 | 0 | 800 | 0.00 | 100.0% |
| Basidiospores | 13 | 2987 | 0.004 | 0 | 3467 | 0.00 | 100.0% |
| [symbol] | 7 | 0 | GIO | 0 | 27 | 0.00 | 100.0% |
| [symbol] | | | | | | | |
| Cladosporium | 53 | 2267 | 0.023 | 7 | 4533 | 0.00 | 99.8% |
| Epicoccum | 0 | 13 | 0.00 | 0 | 60 | 0.00 | 100.0% |
| Other Colorless | 0 | 80 | 0.00 | 0 | 47 | 0.00 | 100.0% |
| Other Brown | 13 | 0 | GIO | 7 | 27 | 0.26 | 73.6% |
| Penicillium/ | 13 | 267 | 0.049 | 40 | 1233 | 0.03 | 94.8% |
| Aspergillus | | | | | | | |
| Smuts, etc. | 13 | 187 | 0.07 | 0 | 7 | 0.00 | 100.0% |
| [symbol] | 7 | 0 | GIO | 0 | 0 | NA | 100.0% |
| Torula | 0 | 13 | 0.00 | 0 | 20 | 0.00 | 100.0% |
| Cercospora | 0 | 13 | 0.00 | 0 | 20 | 0.00 | 100.0% |
| Spegazzinia | 0 | 7 | 0.00 | 0 | 0 | NA | 100.0% |
| Trichoderma | 0 | 93 | 0.00 | 0 | 0 | NA | 100.0% |
| Total | 119 | 6114 | 0.019 | 54 | 10241 | 0.01 | 99.3% |

C = Concentration (Mold Spores per cubic meter) i = inside, b = before, o = outside, a = after
SRE = Spore Removal Efficiency
Bold Indicates growth inside the building
NA = Not Appropriate
GIO = Growing inside Only

The foregoing case studies illustrate the effectiveness of the present invention is eliminating both toxic and non-toxic mold infestations from domestic and commercial buildings. This innovative process, consisting of inspection, sampling, designing a unique, site-specific treatment, treatment with specific essential oil blends, and objectively measuring the results, comprises a unique, well-defined protocol for addressing indoor mold-infestation problems. The present inventors know of no other method for removing mold that is cost-effective, complete safe, uses non-hazardous chemicals, and provides long-lasting relief from repeat flare-ups of mold.

We claim:

1. A method for remediation of mold in a building comprising the steps of:
investigating the building to determine a presence of unwanted mold;
sampling said unwanted mold both inside and outside of said building to determine if a source of mold is present in the building, and to determine a species of said mold and an indoor to outdoor ratio of mold spores for said species, an airborne spore concentration for said species, and a number of colony forming units for said species;
diffusing essential oils into the building for a predetermined period of time, where said predetermined period of time is based on a determination of three measures of: said species; said ratio of indoor to outdoor mold spores; said spore concentration; and said number of colony forming units; and
directly applying a household cleaner with essential oils directly to any mold-affected areas.

2. The method of claim 1, wherein the ratio of indoor to outdoor mold spores is between 1 and 2 and wherein the number of colony forming units is 500 or less, then the diffusing step lasts for 24 continuous hours.

3. The method of claim 1, wherein the ratio of indoor to outdoor mold spores is between 2 and 10 and wherein the number of colony forming units is between 500 and 1000, then the diffusing step lasts for 36 continuous hours.

4. The method of claim 1, wherein the ratio of indoor to outdoor mold spores is greater than 10 and wherein the number of colony forming units is greater than 1000, then the diffusing step lasts for 48 continuous hours.

5. The method of claim 1, wherein the mold species is only found inside, and the airborne spore concentration in spores/m³ air is between 1 and 10 and wherein the number of colony forming units is 500 or less, then the diffusing step lasts for 24 continuous hours.

6. The method of claim 1, wherein the mold species is only found inside, and the airborne spore concentration in spores/m³ air is between 10 and 100 and wherein the number of colony forming units is between 500 and 1000, then the diffusing step lasts for 36 continuous hours.

7. The method of claim 1, wherein the mold species is only found inside, and the airborne spore concentration in spores/m³ air is greater than 1000 and wherein the number of colony forming units is greater than 1000, then the diffusing step lasts for 48 continuous hours.

8. The method of claim 1, wherein species of mold is determined to be at least one of Stachybotrys, Aspergillus/Penicillium, Acremonium, Cladosporium, Alternaria, Chaetomium Memnoniella, and Curvularia, and wherein the airborne spore concentration in spores/m³ air is between 1 and 10 and wherein the number of colony forming units is 500 or less, then the diffusing step lasts for 24 continuous hours.

9. The method of claim 8 wherein the step of directly applying an undiluted household cleaner with essential oils is preceded by supplementing the undiluted household cleaner with undiluted essential oils.

10. The method of claim 1, wherein species of mold is determined to be at least one of Stachybotrys, Aspergillus/Penicillium, Acremonium, Cladosporium, Alternaria, Chaetomium Memnoniella, and Curvularia, and wherein the airborne spore concentration in spores/m³ air is between 10 and 100 and wherein the number of colony forming units is between 500 and 1000, then the diffusing step lasts for 48 continuous hours.

11. The method of claim 10 wherein the step of directly applying an undiluted household cleaner with essential oils is preceded by supplementing the undiluted household cleaner with undiluted essential oils.

12. The method of claim 1, wherein species of mold is determined to be at least one of Stachybotrys, Aspergillus/Penicillium, Acremonium, Cladosporium, Alternaria, Chaetomium Memnoniella, and Curvularia, and wherein the airborne spore concentration in spores/m³ air is greater than 1000 and wherein the number of colony forming units is greater than 1000, then the diffusing step lasts for 72 continuous hours.

13. The method of claim 12 wherein the step of directly applying an undiluted household cleaner with essential oils is preceded by supplementing the undiluted household cleaner with undiluted essential oils.
